(19) United States
(12) Patent Application Publication
(10) Pub. No.: US 2026/0102378 A1
(43) Pub. Date: Apr. 16, 2026

(54) Therapeutic and Topical Anti-Itch Composition

(71) Applicant: Edward Becker, Lincoln Park, NJ (US)

(72) Inventor: Edward Becker, Lincoln Park, NJ (US)

(21) Appl. No.: 19/354,043

(22) Filed: Oct. 9, 2025

Related U.S. Application Data
(60) Provisional application No. 63/705,567, filed on Oct. 10, 2024.

(51) Int. Cl. A61K 31/428; A61K 9/00; A61K 9/08; A61K 31/425; A61K 47/02; A61K 47/10; A61K 47/18; A61K 47/20

(52) U.S. Cl. CPC A61K 31/428; A61K 9/0014; A61K 9/08; A61K 31/425; A61K 47/02; A61K 47/10; A61K 47/18; A61K 47/183; A61K 47/20

(57) ABSTRACT

An anti-itch composition in the form of a gelatinous or semi-solid dish soap base for providing instant relief from poison ivy-induced itching, redness, soreness, and swelling. The composition is novel, easy-to-use, and includes both natural and synthetic components for cleansing, antimicrobial protection, and skin conditioning. In one embodiment, the anti-itch composition comprises a solution of water, a plurality of surfactants, a pH adjuster, a fragrance, a chelating agent, a plurality of antimicrobial agents, and a colorant. The formulation rapidly dries within 1-3 minutes of application, forming a thin protective layer on the skin to reduce irritation and inflammation while preventing secondary infections. The composition can be topically applied to affected body areas by users of all genders and ages, and offers a safe, effective, and convenient alternative to traditional medication-based treatments for poison ivy rashes.

### CROSS-REFERENCE TO RELATED APPLICATION

The present application claims priority to, and the benefit of, U.S. Provisional Application No. 63/705,567, which was filed on Oct. 10, 2024, and is incorporated herein by reference in its entirety.

### FIELD OF THE INVENTION

The present invention generally relates to the field of skincare products. More specifically, the present invention relates to a novel topical anti-itch composition for providing relief from poison ivy-induced skin ailments such as itching, redness, soreness, and swelling. The anti-itch composition has a formulation combining water-based components, surfactants, and natural oil-based emollients to deliver cleansing, antimicrobial protection, and moisturizing effects. The composition is preferably in the form of a gelatinous or semi-solid dish soap base and can be conveniently applied to any affected area of the body. Upon application, the composition dries within minutes, forming a soothing protective layer that relieves irritation, prevents secondary infections, and leaves the skin hydrated and comfortable. Accordingly, the present disclosure makes specific reference thereto. Nonetheless, it is to be appreciated that aspects of the present invention are also equally applicable to other like applications, devices, and methods of manufacture.

### BACKGROUND

By way of background, poison ivy exposure is a common cause of allergic contact dermatitis which results from direct skin contact with urushiol, the oily resin present in the plant. Individuals who contract poison ivy may experience a variety of ailments including severe itching, irritation, redness, swelling, and blistering. The symptoms often persist for several days and can significantly interfere with daily activities.

Constant scratching of the affected area may worsen the condition, causing the skin to break and leading to oozing, open sores, and an increased risk of secondary bacterial infection. Traditional remedies often provide limited relief or require prolonged application before noticeable results are achieved. Accordingly, individuals desire a convenient and effective topical composition that can be applied directly to poison ivy-affected areas to instantly relieve itching, reduce redness and swelling.

Therefore, there exists a long-felt need in the art for a topical composition for providing improved protection against irritation, redness, and swelling caused by poison ivy exposure. There is also a long-felt need in the art for an improved anti-itch formulation that instantly relieves itching. Additionally, there is a long-felt need in the art for a topical product that forms a protective barrier over the affected skin to minimize secondary infections. Moreover, there is a long-felt need in the art for a composition that moisturizes and soothes irritated skin while counteracting the drying effects of surfactants. Further, there is a long-felt need in the art for a topical anti-itch product that is easy to apply, dries quickly, and is absorbed effectively into the skin. Furthermore, there is a long-felt need in the art for a safe, non-prescription composition that can be applied to different parts of the body to provide relief from poison ivy symptoms. Finally, there is a long-felt need in the art for a composition that combines cleansing, antimicrobial, and moisturizing properties in a simple, convenient formulation with pleasant sensory characteristics.

The subject matter disclosed and claimed herein, in one embodiment thereof, comprises an anti-itch composition for preventing and relieving itching, redness, soreness, and swelling caused by poison ivy contact. The composition comprises a plurality of surfactants selected from one or more of the group consisting of sodium lauryl sulfate (SLS) (C₁₂H₂₅NaO₄S), sodium laureth sulfate (SLES), C10-16 Alkyldimethylamine Oxide, C9-11 Pareth-8, sodium C12-15 alketh sulfate, lauramidopropylamine oxide, and poloxamer 124, a pH adjuster (i.e., sodium chloride), a chelating agent (i.e., tetrasodium glutamate diacetate [GLDA]), a plurality of preservatives, and purified water as a carrier base, wherein the composition is in the form of a gelatinous or semi-solid dish soap. The composition can further include stabilizers, antimicrobial agents, moisturizing agents, fragrances, and colorants to enhance performance. When applied to the skin, the composition provides instant relief from itch, reduces inflammation, and forms a thin protective layer that calms irritation and minimizes the risk of secondary infection.

In this manner, the novel therapeutic anti-itch composition of the present invention accomplishes all of the foregoing objectives and provides users with a relatively safe, easy, cost-effective, and effective solution for addressing poison ivy-induced skin irritation. The composition combines cleansing agents to remove urushiol, antimicrobial agents to prevent infection, and natural emollients and moisturizers to soothe and hydrate the skin. The formulation dries quickly, is convenient to use, and can be applied to any affected area of the body. The composition improves skin care routines by delivering rapid, reliable, and long-lasting relief from poison ivy discomfort without the need for prescription medications.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an anti-itch composition for providing instant relief from poison ivy-induced itching, redness, soreness, and swelling. The composition is in the form of a gelatinous or semi-solid dish soap base and includes a composition or solution of water as a carrier base, a plurality of surfactants, stabilizers, antimicrobial agents, moisturizers, pH adjusters, fragrances, and colorants. The composition is configured for topical application on skin to prevent inflammation and irritation and to alleviate itchiness associated with poison ivy exposure.

In a further embodiment of the present invention, a method of treating poison ivy-induced itch is disclosed, the method comprising the steps of contacting an affected area of a user's skin with an anti-itch composition, wherein the composition comprises, by weight, 6-12% sodium laureth sulfate, 3-5% sodium chloride, 2-5% lauramidopropylomine oxide, 1-4% fragrance, 1-4% poloxamer 124, 0-4% tetrasodium glutamate diacetate (GLDA), 0-4% methylisothiazolinone, 0-4% benzisothiazolinone, 0-4% colorants, wherein the balance comprises water and additional stabilizers, preservatives, and functional agents, and enabling the composition to dry on the skin within 1-3 minutes to form a thin protective layer that reduces irritation and prevents further spread of the poison ivy rash.

In yet another embodiment of the present invention, a composition of use as a soothing anti-itch gel comprises, by weight of the composition: 6-12% sodium C12-15 alketh sulfate, 3-5% sodium chloride, 2-5% lauramidopropylomine oxide, 1-4% fragrance, 1-4% poloxamer 124, 0-4% tetrasodium glutamate diacetate (GLDA), 0-4% methylisothiazolinone, 0-4% benzisothiazolinone, and 0-4% colorants. The composition is blended with water to create a topical semi-solid formulation that calms irritation, moisturizes skin, and provides antiseptic protection.

The anti-itch composition of the present invention does not require animal testing and comprises primarily natural and organic components, including olive oil, coconut oil, and terpineol. The composition is preferably semi-transparent, readily absorbed into the skin, and provides natural healing and skin conditioning without the use of steroids or prescription drugs.

The advantage of the anti-itch composition of the present invention is that it provides users with an improved topical product designed to deliver enhanced protection against irritation, redness, soreness, and swelling from poison ivy exposure. The ingredients act synergistically to deliver cleansing, antimicrobial, film-forming, and moisturizing effects. The composition can be topically applied at room temperature and dries within 1-3 minutes, leaving behind a protective, soothing barrier. A user may apply a small amount of the composition to poison ivy-exposed skin regions to reduce inflammation, prevent infection, and achieve instant relief from itching.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of the components of the anti-itch composition of the present invention to provide relief from poison ivy-induced itching, redness, soreness, and swelling;

FIG. 2 illustrates a perspective view showing the topical application of the anti-itch composition of the present invention onto an affected area in accordance with one embodiment of the disclosure; and

FIG. 3 illustrates another embodiment of the container in which the anti-itch formulation composition can be available for consumers.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It can be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments can be combined.

As noted above, there is a long-felt need in the art for a topical composition for providing improved protection against irritation, redness, and swelling caused by poison ivy exposure. There is also a long-felt need in the art for an improved anti-itch formulation that instantly relieves itching. Additionally, there is a long-felt need in the art for a topical product that forms a protective barrier over the affected skin to minimize secondary infections. Moreover, there is a long-felt need in the art for a composition that moisturizes and soothes irritated skin while counteracting the drying effects of alcohol and surfactants. Further, there is a long-felt need in the art for a topical anti-itch product that is easy to apply, dries quickly, and is absorbed effectively into the skin. Furthermore, there is a long-felt need in the art for a safe, non-prescription composition that can be applied to different parts of the body to provide relief from poison ivy symptoms. Finally, there is a long-felt need in the art for a composition that combines cleansing, antimicrobial, and moisturizing properties in a simple, convenient formulation with pleasant sensory characteristics.

The present invention, in one exemplary embodiment, is a method of treating poison ivy-induced itch, the method comprising the steps of contacting an affected area of a user's skin with an anti-itch composition, wherein the composition comprises, by weight, 6-12% sodium laureth sulfate or sodium C12-15 alketh sulfate, 2-5% sodium chloride, 2-5% lauramidopropylomine oxide, 1-4% fragrance, 1-4% poloxamer 124, 0-4% tetrasodium glutamate diacetate (GLDA), 0-4% methylisothiazolinone, 0-4% benzisothiazolinone, 0-4% colorants, wherein the balance comprises water and additional stabilizers, preservatives, and functional agents, and functional agents, and enabling the composition to dry on the skin within 1-3 minutes to form a thin protective layer that reduces irritation and prevents further spread of the poison ivy rash.

The present invention, in another exemplary embodiment, is a method of treating poison ivy-induced itch, the method comprising the steps of contacting an affected area of a user's skin with an anti-itch composition, wherein the composition comprises, sodium laureth sulfate or sodium C12-15 alketh sulfate, sodium chloride, lauramidopropylamine oxide, fragrance, poloxamer 124, tetrasodium glutamate diacetate (GLDA), methylisothiazolinone, benzisothiazolinine, colorants, and wherein the balance comprises water and additional stabilizers, preservatives, and functional agents, and enabling the composition to dry on the skin within 1-3 minutes to form a thin protective layer that reduces irritation and prevents further spread of the poison ivy rash.

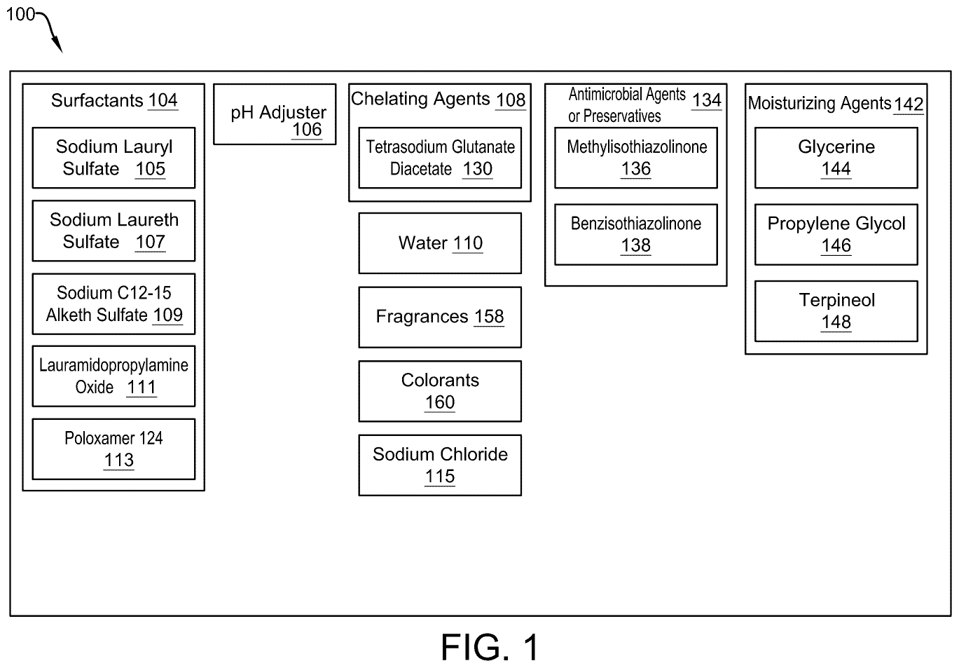

Referring initially to the drawings, FIG. 1 illustrates a diagrammatic representation of the components of the anti-itch composition of the present invention to provide relief from poison ivy-induced itching, redness, soreness, and swelling. The anti-itch composition or solution 100 of the present invention is preferably a topical anti-itch composition in the form of a gelatinous or semi-solid dish soap base to provide instant relief from poison ivy-induced itching, redness, soreness, and swelling. The anti-itch composition or solution 100 can include surfactants 104, pH adjuster 106, chelating agent 108, water 110 as a base, fragrances 158, preservatives or antimicrobial agents 134, and colorants 160, as the core ingredients.

One or more sulfates function as surfactants and are combined with water softeners, and pH adjusters or buffers. The surfactants 104 improve the consistency of the composition or solution 100 and performance thereof by preventing precipitation of surfactants and maintaining balanced alkalinity. The surfactants 104 are selected from one or more of the group consisting of sodium lauryl sulfate (SLS) (C₁₂H₂₅NaO₄S) 105, sodium laureth sulfate (SLES) 107, sodium C12-15 alketh sulfate 109, lauramidopropylamine oxide 111, and poloxamer 124 113.

Purified water 110 is the primary solvent and carrier for the composition or solution 100 and provides a medium for dissolving surfactants, salts, and other functional ingredients of the composition or solution 100. Purified water 110 provides gelatinous consistency and helps regulate viscosity and spreadability of the composition or solution 100.

The composition or solution 100 can include a plurality of surfactants 104 which function as cleansing and lathering agents. The surfactants 104 can comprise sodium lauryl sulfate (SLS) (C₁₂H₂₅NaO₄S) 105 which is a primary anionic surfactant to produce foam. SLS 105 has high foaming, detergent, and emulsifying properties which improve cleansing action and help removing urushiol, the oily toxin present in poison ivy. SLS 105 also reduces surface tension of the composition or solution 100 to enable deep penetration of the composition or solution 100 into the skin of a human.

Sodium laureth sulfate (SLES) 107 or sodium C12-15 alketh sulfate 109 provides lathering and cleansing functions as a surfactant and works to increase distribution and spreadability of the composition or solution 100. SLES 107 also improves skin compatibility of the composition or solution 100 while maintaining strong detergent properties thereof.

Sodium chloride 115 functions as a pH adjuster or buffer and provides a topical treatment in a hypertonic solution to help reduce swelling and promote wound healing through a process called osmosis. Applied to the skin or eyes, the high salt concentration draws fluids out of cells and tissues, helping to cleanse the area and draw out bacteria. Sodium chloride 115 functions as a pH adjuster or buffer to maintain the optimal acidity and alkalinity balance of the composition or solution 100. Sodium chloride 115 stabilizes various surfactants and minimizes skin irritation. The pH adjuster can further be selected from one or more of the group consisting of Sodium chloride, Sodium hydroxide, and Sodium Cumene-Sulfonate.

C10-16 Alkyldimethylamine Oxide or lauramidopropylamine oxide 111 functions as an amphoteric surfactant and provides foam stabilization and thickening, giving the composition or solution 100 a semi-solid appearance. Lauramidopropylamine oxide 111 also provides mild antimicrobial activity and supports cleansing of oils and residues.

Poloxamer 124 113 is a type of nonionic surfactant that is commonly used in cosmetics and personal care products. This water-soluble polymer helps in emulsifying and stabilizing formulations, improving the texture, and enhancing the appearance of products. Poloxamer 124 113 is often used in skincare products such as creams, lotions, and serums, as well as in some makeup products and hair care products. It is known for its ability to create a smooth, silky texture and its non-irritating and non-sensitizing properties. The chemical formula of Poloxamer 124 is C₅H₁₀O₂.

The composition or solution 100 can include a plurality of stabilizers or chelating agents 108 to control viscosity, film formation, and stability of the anti-itch composition or solution 100. The chelating agents 108 can include tetrasodium glutamate diacetate 130 which functions as a humectant and skin-conditioning agent. Tetrasodium glutamate diacetate 130 helps retain moisture within the composition 100, preventing excessive drying of the skin. Tetrasodium glutamate diacetate 130 is easily soluble in the carrier water 110 and is compatible with surfactants and emollients used in the composition or solution 100. Tetrasodium Glutamate Diacetate (C₉H₉NNa₄O₈) 130 functions as a chelating agent and binds to and inactivates trace metal ions (i.e., iron, calcium, magnesium) to prevent destabilization of the composition or solution 100. Tetrasodium Glutamate Diacetate (GLDA) 130 also improves the performance of antimicrobial agents or preservatives of the composition or solution 100 and helps maintain the gelatinous consistency and prevents precipitation or cloudiness of the composition or solution 100.

The composition or solution 100 can include a plurality of antimicrobial agents or preservatives 134 selected from the group including phenoxyethanol, methylisothiazolinone 136, and benzisothiazolinone 138 which prevents microbial contamination of the composition or solution 100 from bacteria, yeasts, and molds, during storage and use. The preservatives 134 provide synergistic action with the other ingredients included in the composition or solution 100 for improved antimicrobial efficacy.

Methylisothiazolinone 136 is used in micro-concentrations such as less than 0.01% by weight and can be included to increase shelf-life of the composition or solution 100 by preventing microbial growth. Methylisothiazolinone 136 preferably provides protection against bacteria and fungi and works together with the other ingredients for maintaining safety of the composition or solution 100.

Benzisothiazolinone 138 can be included in the composition or solution 100 to provide broad-spectrum antiseptic and disinfectant properties, especially against Gram-positive bacteria and fungi. Benzisothiazolinone 138 also protects irritated or broken skin from secondary bacterial infections and provides soothing effect to the skin when the composition or solution 100 is applied thereon.

The composition or solution 100 can include a plurality of moisturizing agents 142 to make the application of the composition or solution 100 soothing and gentle. Glycerin 144 functions as humectant and keeps the skin hydrated to reduce dryness and flaking caused by poison ivy rashes. Further, glycerin 144 provides smooth and uniform texture to the composition or solution 100 and soothe irritation caused by surfactants used in the composition or solution 100.

Propylene Glycol 146 functions as a humectant, solvent, and penetration enhancer for the composition or solution 100. Propylene Glycol 146 dissolves active ingredients and fragrances into the composition or solution 100 and improves skin absorption of natural oils and antimicrobial agents.

Terpineol 148 has natural terpene alcohol and provides skin-conditioning and soothing effects. Further, Terpineol 148 has a pine-like fragrance which improves the sensory effect of the composition or solution 100. Terpineol 148 also provides mild antimicrobial properties, thereby adding additional protection against infection caused by poison ivy.

The composition or solution 100 can include a plurality of pH adjusters 150 for maintaining stability, consistency, and skin compatibility in the anti-itching composition or solution 100. Sodium chloride 115 also functions as a viscosity modifier and maintains the gel-like or semi-solid consistency of the composition or solution 100. Sodium chloride 115 also contributes to osmotic balance of the composition or solution 100 on the skin to reduce irritation potential.

The composition or solution 100 can include one or more fragrances 158 to provide a scent profile that masks any chemical or medicinal odors from the base composition. The fragrances 158 can be blended with natural fragrance contributors such as terpineol 148 to balance aroma of the composition or solution 100. Fragrances 158 may include blends of aromatic compounds, terpineol, esters, aldehydes, and essential oil or derivatives.

One or more colorants 160 to provide a color to the composition or solution 100 can be included in the composition or solution 100. The colorants 160 may include water soluble dyes such as Blue 1 (FD&C Blue No. 1), Yellow 5 (FD&C Yellow No. 5), and Red 33 (D&C Red No. 33). All the colors are FDA-approved for topical use in controlled concentrations in the composition or solution 100.

Referring to Table 1, there is disclosed an anti-itching composition with a number of different components in varying quantities in order to achieve a desired result of rich foam and strong cleansing to remove urushiol oil. The anti-itching composition or solution 100, in one potential embodiment, is provided as a gelatinous or semi-solid dish soap and provides antiseptic and anti-inflammatory relief while forming a thin film barrier. One such exemplary embodiment of the anti-itching composition or solution 100 is provided as shown in Table 1.

TABLE 1

| | Composition in percentage |
|---|---|
| Component | by weight (% w/w) |
| Water | 50-60 |
| Sodium Laureth Sulfate (SLES) or | 6-12 |
| Sodium C12-15 alketh sulfate | |
| Sodium chloride | 2-5 |
| Lauramidopropylomine oxide | 2-5 |
| Fragrance | 1-4 |
| Poloxamer 124 | 1-4 |
| Tetrasodium glutamate diacetate | 0-4 |
| Methylisothiazolinone | 0-4 |
| Benzisothiazolinone | 0-4 |
| Colorants | 0-4 |

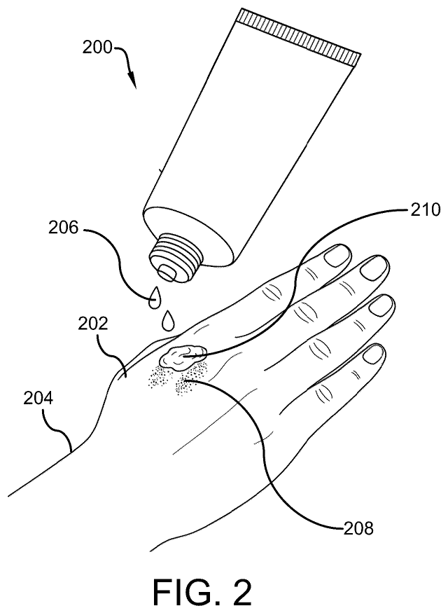

FIG. 2 illustrates a perspective view showing the topical application of the anti-itch composition of the present invention onto an affected area in accordance with one embodiment of the disclosure. As illustrated, the composition or solution 100 is contained within a dispensing tube 200, which is oriented above the skin surface 202 of a human hand 204. A plurality of droplets 206 of the anti-itch gel composition or solution 100 is applied on the rash region 208 (i.e., representing poison ivy exposure). Upon contact, the composition or solution 100 spreads to form a gel layer 210 that soothes the affected area.

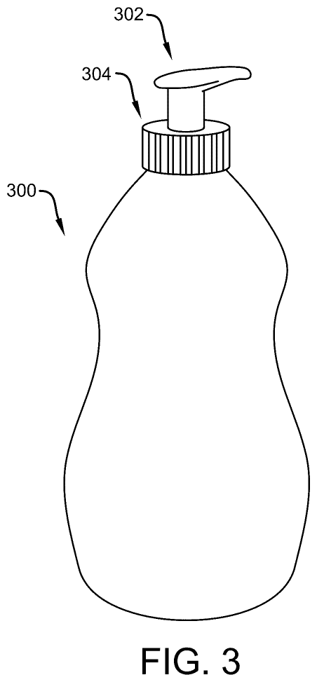

FIG. 3 illustrates another embodiment of the container in which the anti-itch formulation composition can be available for consumers. In the present embodiment, the composition or solution 100 is stored in a squeezable or pump-style bottle 300 with a dispensing cap 302 at the upper end 304 thereof. The inner volume of the container 300 stores the gelatinous or semi-solid composition or solution 100. As shown, the design of the container 300 enables controlled dispensing of the semi-solid composition or solution 100 for direct application to a user's skin.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “topical anti-itch composition”, “gelatinous anti-itch formulation”, “anti-itch composition”, and “composition” are interchangeable and refer to the topical anti-itch composition or solution 100 of the present invention.

Notwithstanding the forgoing, the therapeutic topical anti-itch composition or solution 100 of the present invention can contain any additional ingredient known in the art without affecting the overall concept of the invention, provided that it accomplishes the above-stated objectives. One of ordinary skill in the art will appreciate that the ingredients and composition of the therapeutic topical anti-itch composition or solution 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A skin treatment and anti-itch solution comprising:
a solution comprising water, a plurality of surfactants, a pH adjuster, a fragrance, a chelating agent, a plurality of antimicrobial agents, and a colorant;
wherein said plurality of surfactants selected from one or more of the group consisting of sodium laureth sulfate (SLES), sodium C12-15 alketh sulfate, lauramidopropylamine oxide, and poloxamer 124; and
further wherein said plurality of antimicrobial agents selected from one or more of the group consisting of Phenoxyethanol, Methylisothiazolinone, and benzisothiazolinine.

2. The skin treatment and anti-itch solution of claim 1, wherein said chelating agent is Tetrasodium Glutamate Diacetate (C₉H₉NNa₄O₈).

3. The skin treatment and anti-itch solution of claim 2, wherein said plurality of antimicrobial agents selected form one or more of the group consisting of methylisothiazolinone and benzisothiazolinine.

4. The skin treatment and anti-itch solution of claim 2 further comprising a plurality of moisturizing agents selected from one or more of the group consisting of Glycerin, Propylene Glycol, and Terpineol.

5. The skin treatment and anti-itch solution of claim 3, wherein said pH adjuster selected from one or more of the group consisting of Sodium chloride, Sodium hydroxide, and Sodium Cumene-Sulfonate.

6. The skin treatment and anti-itch solution of claim 5, wherein said fragrance selected from one or more of the group consisting of a terpineol, an ester, an aldehyde, and an essential oil.

7. The skin treatment and anti-itch solution of claim 6, wherein said colorant selected from one or more of the group consisting of Blue 1 (FD&C Blue No. 1), Yellow 5 (FD&C Yellow No. 5), and Red 33 (D&C Red No. 33).

8. The skin treatment and anti-itch solution of claim 7, wherein a percent weight by weight of: said water is in a range of 50-60, said sodium laureth sulfate (SLES) is in a range of 6-12, said sodium chloride is in a range of 2-5, said lauramidopropylomine oxide is in the range of 2-5, said fragrance is in the range of 1-4, said poloxamer 124 is in the range of 1-4, said Tetrasodium Glutamate Diacetate is in a range of 0-4, said Methylisothiazolinone is in a range of 0-4, said benzisothiazolinone is in the range of 0-4, and said colorants are in a range of 0-4.

9. The skin treatment and anti-itch solution of claim 7, wherein a percent weight by weight of: said water is in a range of 50-60, said sodium C12-15 alketh sulfate is in a range of 6-12, said sodium chloride is in a range of 2-5, said lauramidopropylomine oxide is in the range of 2-5, said fragrance is in the range of 1-4, said poloxamer 124 is in the range of 1-4, said Tetrasodium Glutamate Diacetate is in a range of 0-4, said Methylisothiazolinone is in a range of 0-4, said benzisothiazolinone is in the range of 0-4, and said colorants are in a range of 0-4.

10. A method of treating poison ivy-induced itch, the method comprising the steps of:
contacting an affected area of a user's skin with an anti-itch composition, wherein the composition comprises, water, a plurality of surfactants, sodium chloride, fragrance, tetrasodium glutamate diacetate (GLDA), methylisothiazolinone, benzisothiazolinine, and colorants; and
applying said anti-itch composition to said affected area of the user's skin.

11. The method of treating poison ivy-induced itch of claim 10, wherein said plurality of surfactants selected from one or more of the group consisting of sodium laureth sulfate (SLES), sodium C12-15 alketh sulfate, lauramidopropylamine oxide, and poloxamer 124.

12. The method of treating poison ivy-induced itch of claim 10 further comprising a step of drying said anti-itch composition on said affected area of the user's skin.

13. The method of treating poison ivy-induced itch of claim 12 further comprising a step of forming a protective layer of said anti-itch composition over said affected area of the user's skin.

14. The method of treating poison ivy-induced itch of claim 11 further comprising the steps of:
drying said anti-itch composition on said affected area of the user's skin; and
forming a protective layer of said anti-itch composition over said affected area of the user's skin.

15. A method of treating poison ivy-induced itch, the method comprising the steps of:
contacting an affected area of a user's skin with an anti-itch composition, wherein said anti-itch composition comprises a solution of water, a plurality of surfactants, a pH adjuster, a fragrance, a chelating agent, a plurality of antimicrobial agents, and a colorant;
wherein said plurality of surfactants selected from one or more of the group consisting of sodium laureth sulfate (SLES), sodium C12-15 alketh sulfate, lauramidopropylamine oxide, and poloxamer 124;
applying said anti-itch composition to said affected area of the user's skin;
drying said anti-itch composition on said affected area of the user's skin; and
forming a protective layer of said anti-itch composition over said affected area of the user's skin.

16. The method of treating poison ivy-induced itch of claim 15, wherein said chelating agent is tetrasodium glutamate diacetate (GLDA).

17. The method of treating poison ivy-induced itch of claim 16, wherein said plurality of antimicrobial agents selected from one or more of the group consisting of methylisothiazolinone and benzisothiazolinone.

18. The method of treating poison ivy-induced itch of claim 17, wherein said pH adjuster is Sodium chloride.

19. The method of treating poison ivy-induced itch of claim 18, wherein said fragrance selected from one or more of the group consisting of a terpineol, an ester, an aldehydes, and an essential oil.

20. The method of treating poison ivy-induced itch of claim 19, wherein said colorant selected from one or more of the group consisting of Blue 1 (FD&C Blue No. 1), Yellow 5 (FD&C Yellow No. 5), and Red 33 (D&C Red No. 33).
